(19) United States
(12) Patent Application Publication
(10) Pub. No.: US 2011/0045067 A1
(43) Pub. Date: Feb. 24, 2011

(54) SOFT GEL CAPSULES

(75) Inventors: Ingvild Johanne Haug, Tromso (NO); Kurt Ingar Draget, Tromso (NO)

Correspondence Address: IPHORGAN, LTD., 1130 LAKE COOK ROAD, SUITE 240, BUFFALO GROVE, IL, 60089, US

(73) Assignee: AYANDA AS, Tromso (NO)

(21) Appl. No.: 12/864,631

(22) PCT Filed: Jan. 29, 2009
(86) PCT No.: PCT/GB2009/000248

§ 371 Date: Oct. 6, 2010

(30) Foreign Application Priority Data
Jan. 29, 2008 (GB) 0801643.8

(51) Int. Cl. A61K 9/64; A61K 35/12; A61P 43/00

(52) U.S. Cl. 424/456; 424/520

(57) ABSTRACT

The invention relates to soft gelatin capsules containing at least one pharmaceutical, wherein the capsule shell includes carrageenan and cold water fish gelatin or a mixture of cold fish gelatin and warm fish gelatin.

This invention relates to soft gel capsules for oral administration of nutraceuticals or pharmaceuticals (hereinafter jointly referred to as pharmaceuticals), as well as to a process for their production and the gel mixture used for their production.

Many pharmaceuticals, e.g. analgesics, fish oils, plant oils, etc., are administered encapsulated by a gelatin case. In this way, the correct dosage may be regulated and the taste of the pharmaceutical is masked.

Gelatin is a mixture of water-soluble proteins produced by collagen tissue hydrolysis followed by gelatin extraction at elevated temperature. The starting tissue material is typically animal waste such as skin, bones, connective tissue, and the like.

Most of the gelatin used for product encapsulation has tended to be mammalian gelatin, in particular bovine or porcine gelatin, which has particularly suitable gel properties for encapsulation and oral administration. Standard encapsulation equipment has been developed for use with mammalian gelatin. Gelatin for encapsulation is referred to as “soft” or “hard”.

Mammalian, and particularly bovine and porcine, products are however unacceptable to many people for moral, religious and health-based reasons.

Thus there has been interest in using fish gelatins in place of mammalian gelatins for producing casings for oral capsules. Thus for example US-A-2003/0232076 discusses the preparation of soft gelatin capsules using fish gelatins. Capsules produced with fish gelatin would generally be “soft”.

So far however, because of the difference in the gelling and melting points of fish gelatins, especially cold water fish gelatins, as compared with those of the standard mammalian gelatins, it has been problematical to use the standard encapsulation equipment to produce soft gel capsules using fish gelatin.

Fish gelatins are generally derived from either cold water fish (for example cod, haddock, hake, pollock, cusk, sole, flounder, turbot, halibut, plaice, lump fish, redfish, pike, trout and salmon) or warm water fish (for example tilapia, shark or carp). These differ in their gelling and melting points, the former having gel points below 15° C., typically 4 to 12° C., and melting points below 22° C., typically 12 to 19° C., while the latter have gel points above 15° C., typically 18 to 24° C., and melting points in the range 22 to 32° C. For present purposes, cold water fish may be taken to mean any species of fish living predominantly in water of 18° C. or below.

Gelatins are also characterized by their Bloom values. The lower the Bloom value, in general the lower the gel point and the lesser the mechanical stability of the resultant gel. Bloom values above 190 may be considered to be high while those below 180 may be considered to be low.

Cold water fish gelatins tend to have low or non-existent Bloom values and such gelatins do not produce gels of sufficient mechanical and thermal stability for pharmaceutical encapsulation. Thus for example US-A-2003/0232076 mentioned above uses Miyagi's MPM Shark fish gelatin, which is a warm water fish gelatin with a Bloom value of 110-140, and a Croda 200B Fish Gelatin with a high Bloom value of 207. The Bloom value 207 gelatin gave the better handling properties. A Bloom value of this magnitude is indicative of the gelatin being from warm water fish.

We have now found, however, that soft gel capsules composed of a significant amount of cold water fish gelatins can be manufactured using standard encapsulation equipment if an amount of warm water fish gelatin is also used. The relative proportion of cold water fish gelatin used may be further increased by the inclusion of a carrageenan, a polysaccharide extracted from seaweed, and more preferably kappa-carrageenan. Particularly preferably, the gel composition also includes a plasticizer, such as for example glycerol or sorbitol.

Thus viewed from one aspect the invention provides a soft'gelatin capsule containing at least one pharmaceutical, wherein the capsule shell comprises carrageenan and cold water fish gelatin.

Viewed from a further aspect the invention provides a soft gelatin capsule containing at least one pharmaceutical, wherein the capsule shell comprises at least 40% wt cold water fish gelatin relative to the total weight of gelatin

Viewed from a still further aspect the invention provides a gelatin capsule shell ingredient composition comprising carrageenan and cold water fish gelatin.

Viewed from a yet further aspect the invention provides a gelatin capsule shell ingredient composition comprising cold water fish gelatin and warm water fish gelatin, wherein said cold water fish gelatin constitutes at least 40% wt of the total gelatin.

Viewed from another aspect the invention provides a process for producing pharmaceutical composition in orally administrable soft capsule form, which process comprises encapsulating a pharmaceutical material within a shell-forming gelatin composition, characterized in that said gelatin composition comprises carrageenan and cold water fish gelatin.

Viewed from another aspect the invention provides a process for producing pharmaceutical composition in orally administrable soft capsule form, which process comprises encapsulating a pharmaceutical material within a shell-forming gelatin composition, characterized in that said gelatin composition comprises cold water fish gelatin as at least 40% wt of the total gelatin.

By cold water fish gelatin is meant herein a gelatin having a gel point of up to 15° C., especially up to 12° C., typically 4-12° C. The Bloom value of the cold water fish gelatin will typically be below 120 or essentially non-existent, or not measurable according to standard procedures. Thus for example the cold water fish gelatin is preferably one for which a 6.67% wt. solution in water will not produce a self-supporting gel at 10° C., i.e. having a G′ value of less than 120 Pa after 18 hours, preferably less than 100 Pa.

Typical species from which such gelatin may come include cod, haddock, hake, pollock, cusk, sole, flounder, halibut, plaice, lump fish, redfish, pike, trout, turbot and salmon. The weight average molecular weight of the cold water fish gelatin will typically be at least 70 kDa, more typically at least 90 kDa, e.g. 100 to 250 kDa.

In a preferred embodiment, the composition of the invention comprises a carrageenan, a stabilizer, a cold water fish gelatin and, optionally, a warm water fish gelatin.

Where carrageenan is used, this is preferably kappa-carrageenan. The carrageenan, if used, is preferably present in the shell material at a content of 0.01 to 2% wt on a dry solids basis, especially 0.05 to 1% wt, particularly 0.06 to 0.5% wt. To ensure gelling, a physiologically tolerable metal salt (e.g. a potassium salt such as potassium chloride for kappa-carrageenan), will generally be included in the shell material. Typically this will be at a concentration (in the aqueous composition from which the capsule shell is formed) of up to 50 mM, especially 10 to 30 mM.

The cold water fish gelatin conveniently constitutes 15 to 100% wt of the total gelatin material in the aqueous composition from which the soft gel capsule shells are formed, especially 20 to 80% wt, more especially 35 to 70% wt, particularly 40 to 65% wt, e.g. at least 55% wt.

The balance of the gelatin used for soft gel capsule formation according to the invention, i.e. the gelatin which is not cold water fish gelatin, may be a warm water fish gelatin, a mammalian gelatin, an avian gelatin or any other gelatin. Thus, in addition to the cold water fish gelatin, the gelatin used may include warm water fish gelatin, e.g. from fish such as tilapia, carp and shark. The gelatin used however is preferably substantially free of mammalian gelatins, i.e. containing no more than 1%, preferably 0% wt relative to the total gelatin weight. Particularly preferably the gelatin is at least 90% wt, more preferably at least 95% wt, especially 100% wt, fish gelatin.

The Bloom value of the warm water fish gelatin in the total gelatin mixture will typically be at least 180, especially at least 190, more especially 200 to 250. The warm water fish gelatin used preferably has a gel point of 14 to 24° C., especially 18 to 22° C. In a preferred embodiment, the gelatin comprises cold water and warm water fish gelatin in a weight ratio of 99.9:0.1 to 35:65, especially 65:35 to 40:60.

The gelatin conveniently makes up 60 to 80% wt of the shell on a dry solids basis, especially 65 to 75% wt, particularly about 70% wt.

The composition from which the capsule shell is formed preferably also contains a plasticizer, such as for example glycerol or sorbitol. Where present, the plasticizer may comprise up to 35% wt, e.g. 5 to 35% wt, especially 15 to 30% wt, of the shell material on a dry solids basis.

The shell material may optionally include flavours, aromas, colorants and the like as minor components.

The finished capsule may if desired be coated, e.g. with beeswax.

The contents of the capsules may be any pharmaceutical or nutraceutical that is to be delivered orally. The contents will typically be in solid, semi-solid, or liquid form, e.g. powders, gels, emulsions, and liquids. Liquid, emulsion and gel forms are preferred. It is especially preferable that the contents be nutraceuticals, e.g. plant or marine oils such as phospholipids and triglycerides, for example fish oils. Fish oils high in omega-3 fatty acids are especially preferred, e.g. cod or halibut liver oils.

The filled capsules may be prepared by conventional techniques using conventional machinery. Thus for example the shell forming material (with the components in solution/dispersion/emulsion in water) in heated liquid form may be cast into flat ribbons. Pairs of ribbons may be placed between die rolls and dose units of the pharmaceutical/nutraceutical may be injected between the ribbons. On passing between the die rolls the ribbons are fused and the capsules cut out.

Typically the capsules will have a total mass of 250 to 1500 mg, especially 500 to 1000 mg.

By selecting the relative concentrations of the gelatins and the carrageenan, soft capsules may be produced which, will pass through the gastrointestinal tract to release the capsule content at the desired site therein. This allows the uptake of the drug or nutrient to be optimized.

Thus the capsules produced according to the invention are preferably swallowable rather than simply chewable and so preferably are ellipsoidal rather than spherical. As a result, they are preferably seamed and produced by a die roll method, e.g. as described above.

The invention will now be described further with reference to the following non-limiting Examples.

### EXAMPLE 1

### Capsule Shell Composition

| | |
|---|---|
| 24.8 g | cold water fish gelatin* |
| 20.0 g | warm water fish gelatin** |
| 0.2 g | kappa-carrageenan |
| 0.7 g | KCl |
| 20.0 g | glycerol |
| 34.3 g | water |

*gel point 4° C., weight average molecular weight 120 kDa
**gel point 20° C., Bloom value 220

The water, glycerol and kappa-carrageenan are mixed and heated to 90° C. and stirred until the kappa-carrageenan has fully dissolved. The gelatins and the KCl are added, and the mixture is brought to 65-70° C. and stirred until a homogeneous solution, free of solid particles, is formed.

### EXAMPLE 2

### Capsule Shell Composition

| | |
|---|---|
| 31.36 g | cold water fish gelatin * |
| 13.44 g | warm water fish gelatin ** |
| 20.0 g | glycerol |
| 35.2 g | water |

* and ** as in Example 1

The components are mixed and heated to 65-70° C. with stirring until a homogeneous solution, free of solid particles, is formed.

### EXAMPLE 3

### Soft Capsules

Soft capsules are prepared in conventional fashion using the compositions of Examples 1 and 2 for the shells and with a filling of 170 to 1200 mg/capsule (according to capsule size) of cod liver oil.

### EXAMPLE 4

### Soft Capsules

Soft capsules are prepared in conventional fashion using the compositions of Examples 1 and 2 for the shells and with a filling of 50 to 500 mg/capsule (according to capsule size) of paracetamol.

### EXAMPLE 5

### Comparison of Release Profiles

Soft capsules according to Example 4 were compared for their paracetamol release profiles with paracetamol-containing test units having a conventional mammalian gelatin film covering the unit opening.

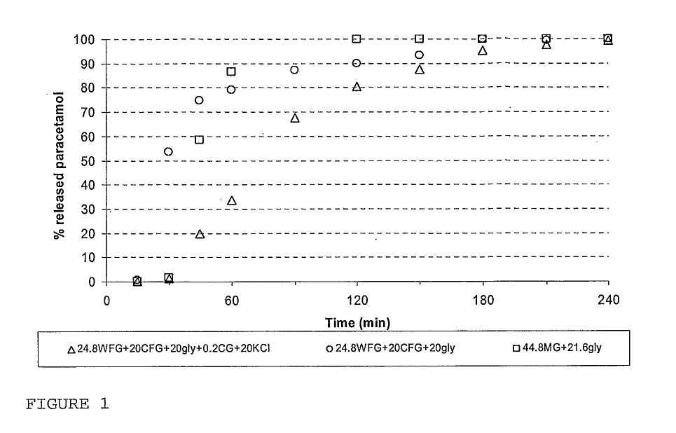

The capsules and test unit were exposed to simulated gastric juice (0.1 M HCl and 0.34M NaCl) at 37° C. and with 60 rpm stirring (according to the European Pharmacopoeia, but without the use of pepsin) in order to study the release of paracetamol. The release profiles are shown in FIG. 1 (the triangle, circle and square symbols represent the data points for the capsules with shells of (i) fish gelatin and kappa-carrageenan, (ii) fish gelatin without kappa-carrageenan, and (iii) test units closed with mammalian gelatin, respectively).

### EXAMPLE 6

### Comparison of Breaking Strengths

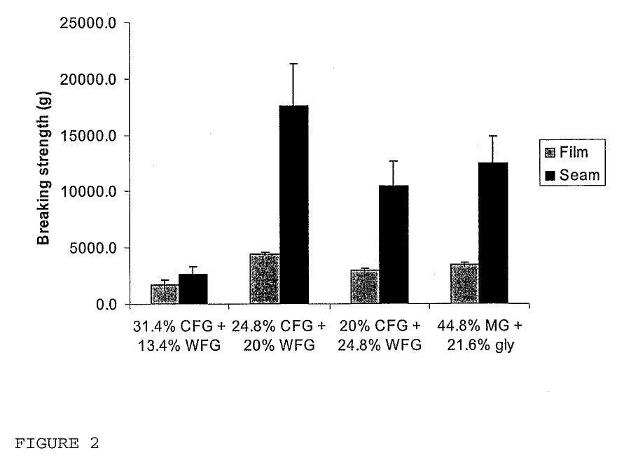

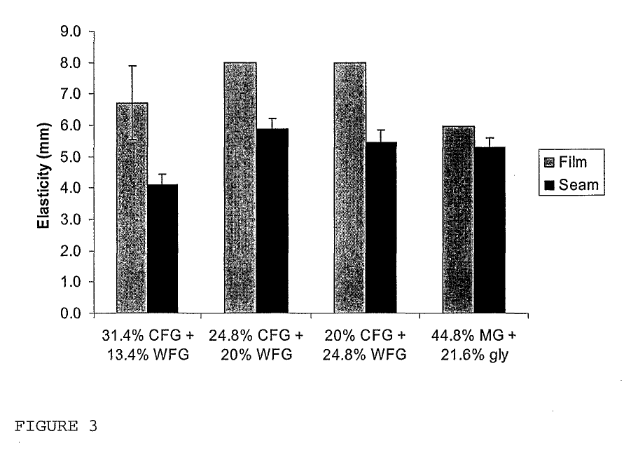

The elasticity and breaking strengths of standard mammalian gelatin soft gel capsules and capsules produced analogously to Example 3 using different ratios of cold and warm water fish gelatins were analysed on a Texture analyzer using the “soft gelatin capsule test” of Stable Microsystem. Tests were performed one month after capsule production. The results (average and standard deviation for a test set of 8 samples) are set out in FIGS. 2 and 3. The black and grey columns are the results for the capsule seams and films respectively. The results, from left to right, are respectively for capsules with 31.4/13.4, 24.8/20 and 20/24.8 weight ratios of cold and warm water fish gelatin mixtures and for a 44.8/21.6 weight ratio mammalian gelatin/glycerol mixture. As can be seen, the mid-range cold/warm water fish gelatin mixture outperforms the others.

1. A soft gelatin capsule containing at least one pharmaceutical, wherein the capsule shell comprises carrageenan and cold water fish gelatin.

2. A soft gelatin capsule containing at least one pharmaceutical, wherein the capsule shell comprises at least 40% wt cold water fish gelatin relative to the total weight of gelatin.

3. A capsule as claimed in either claim 1 wherein the capsule shell comprises cold water fish gelatin and warm water fish gelatin.

4. A capsule as claimed in claim 1 wherein the gelatin in the capsule shell is essentially entirely of piscine origin.

5. A capsule as claimed in claim 1 wherein the capsule shell comprises kappa carrageenan.

6. A capsule as claimed in claim 1 wherein the capsule shell further comprises a plasticizer.

7. A capsule as claimed in claim 1 wherein said pharmaceutical is a fish oil.

8. A capsule as claimed in claim 1 in seamed elongate form.

9. A gelatin capsule shell ingredient composition comprising carrageenan and cold water fish gelatin.

10. A gelatin capsule shell ingredient composition comprising cold water fish gelatin and warm water fish gelatin, wherein said cold water fish gelatin constitutes at least 40% wt of the total gelatin.

11. A composition as claimed in claim 9 comprising a plasticizer.

12. A process for producing pharmaceutical composition in orally administrable soft capsule form, which process comprises encapsulating a pharmaceutical material within a shell-forming gelatin composition, characterized in that said gelatin composition comprises carrageenan and cold water fish gelatin.

13. A process for producing pharmaceutical composition in orally administrable soft capsule form, which process comprises encapsulating a pharmaceutical material within a shell-forming gelatin composition, characterized in that said gelatin composition comprises cold water fish gelatin as at least 40% wt of the total gelatin.

14. A capsule as claimed in claim 2 wherein the capsule shell comprises cold water fish gelatin and warm water fish gelatin.

15. A capsule as claimed in claim 2 wherein the gelatin in the capsule shell is essentially entirely of piscine origin.

16. A capsule as claimed in claim 2 wherein the capsule shell comprises kappa carrageenan.

17. A capsule as claimed in claim 2 wherein the capsule shell further comprises a plasticizer.

18. A capsule as claimed in claim 2 wherein said pharmaceutical is a fish oil.

19. A capsule as claimed in claim 2 in seamed elongate form.

20. A composition as claimed in claim 10 further comprising a plasticizer.
